(19) United States
(12) Patent Application Publication
(10) Pub. No.: US 2016/0095591 A1
(43) Pub. Date: Apr. 7, 2016

(54) MAGNETIC AND/OR HOOK AND LOOP T-TAGS

(71) Applicant: Boston Scientific Scimed, Inc., MAPLE GROVE, MN (US)

(72) Inventors: Paul Smith, Smithfield, RI (US); Samuel Raybin, Marlborough, MA (US); Naroun Suon, Lawrence, MA (US); Tara E. DeLand, Marlborough, MA (US); Justin Elfman, Cherry Hill, NJ (US); Liam K. P. M. Rolle, Boston, MA (US)

(21) Appl. No.: 14/872,739

(22) Filed: Oct. 1, 2015

Related U.S. Application Data
(60) Provisional application No. 62/058,352, filed on Oct. 1, 2014.

(51) Int. Cl. A61B 17/064; A61B 17/08; A61B 17/11; A61B 17/00

(52) U.S. Cl. CPC A61B 17/064; A61B 17/00234; A61B 17/0057; A61B 17/08; A61B 17/1114; A61B 2017/00269; A61B 2017/00278; A61B 2017/00336; A61B 2017/0034; A61B 2017/00606; A61B 2017/00623; A61B 2017/00818; A61B 2017/00876; A61B 2017/081; A61B 2017/1132

(57) ABSTRACT

A device and system for treating tissue includes an anchoring element at a distal end thereof for anchoring the device in a target portion of tissue and a coupling element with a magnet. The anchoring element and the coupling element are coupled to one another by a tether so that, when the anchor element is anchored in a target portion of tissue, the tether passes through the portion of tissue and the coupling element remains outside the portion of tissue. The closing device is configured to couple to a second closing device comprising a second anchoring element tethered to a second coupling element with a magnet to hold the target portion of tissue in a desired location relative to one another.

### PRIORITY CLAIM

The present application claims priority to U.S. Provisional Patent Application Ser. No. 62/058,352 filed Oct. 1, 2014; the disclosure of which is incorporated herewith by reference.

### BACKGROUND

Physicians have been increasingly willing to perform more aggressive interventional and therapeutic endoscopic procedures including, for example, the removal of larger lesions (e.g., cancerous masses), tunneling under the mucosal layer of the gastrointestinal (GI) tract to treat tissue below the mucosa, full thickness removal of tissue, the treatment of issues on other organs by penetrating and passing of the GI tract, and the endoscopic treatment/repair of post-surgical issues (e.g., post-surgical leaks, breakdown of surgical staple lines, anastomic leaks). These procedures my increase the risk of perforating the wall of the GI tract, or may require closure of the GI tract wall as part of the procedure. Endoscopic closure can save costs for the hospital and provide benefits to the patient. However, conventional devices for tissue closure may prove difficult to use and time consuming in certain conditions. In addition, conventional devices may be insufficient to close certain perforations or to treat certain conditions and anatomies such as, for example, large wounds created in the GI tract.

### SUMMARY OF THE DISCLOSURE

The present disclosure is directed to a closing device for treating tissue, comprising an anchoring element at a distal end thereof for anchoring the device in a target portion of tissue and a coupling element, the anchoring element and the coupling element being coupled to one another by a tether so that, when the anchor element is anchored in a target portion of tissue, the tether passes through the portion of tissue and the coupling element remains outside the portion of tissue, wherein the closing device is configured to couple to a second closing device to hold the portion of tissue in a desired location relative to one another.

In an embodiment, the coupling element may be magnetic.

The present disclosure is also direct to a system for treating tissue, comprising a first closing device including a first anchoring element at a distal end thereof for anchoring the first closing device in a first target portion of tissue and a first coupling element, the first anchoring element and the first coupling element being coupled to one another by a first tether so that, when the first anchoring element is anchored in a first target portion of tissue, the first tether passes through the first portion of tissue and the first coupling element remains outside the first portion of tissue; a second closing device including a second anchoring element at a distal end thereof for anchoring the second closing device in a second target portion of tissue and a second coupling element, the second anchoring element and the second coupling element being coupled to one another by a second tether so that, when the second anchoring element is anchored in a second target portion of tissue, the second tether passes through the second portion of tissue and the second coupling element remains outside the second portion of tissue; the first and second coupling elements being configured to couple to one another to hold the first and second portions of tissue in a desired position relative to one another.

In an embodiment, each of the first anchoring elements may include a projection extending transversely to an axis of the tether to resist removal of the first anchoring element from tissue into which it has been inserted.

In an embodiment, the first and second coupling elements are magnetic.

In an embodiment, the disclosure may further include a first sheath with a first tissue piercing distal tip and defining a first lumen therein sized and shaped to receive at least one of the first and second closing devices.

In an embodiment, the first closing device may include a casing surrounding at least a portion of the first coupling element and a portion of the first tether to couple the first tether to the first coupling element.

In an embodiment, there may be a handle coupled to a proximal end of the first sheath and a first actuation mechanism for incrementally deploying the first closing element from the first sheath.

In an embodiment, the first actuation mechanism includes a first actuator on the handle coupled to a first actuation member slidably received in the first sheath to expel the first closing device out of the first sheath upon operation of the first actuator.

In an embodiment, the delivery tool may include a second sheath with a second tissue piercing distal tip and defining a second lumen therein sized and shaped to receive the second closing device while the first closing device is received within the first sheath.

In an embodiment, at least the first and second lumens may include a plurality of further closing devices, each of the further closing devices including a further anchoring element at a distal end thereof for anchoring the further closing device in a further target portion of tissue and a further coupling element, each further anchoring element being coupled to a corresponding further coupling element by an associated further tether so that, when each further anchoring element is anchored in tissue, the associated further tether passes through the tissue and the corresponding further coupling element remains outside the tissue.

In am embodiment, the system may further comprise a second actuation mechanism including a second actuator on the handle coupled to a second actuation member slidably received in the second sheath to push the second closing device out of the second sheath upon operation of the second actuator.

In an embodiment, the first coupling element may include a first hook and loop portion and the second coupling element includes a second hook and loop portion configured to couple to the first hook and loop portion.

In an embodiment, the first and second coupling elements may be shaped to mechanically lock to one another.

In an embodiment, the magnet may be a rare earth magnet.

The present disclosure is also directed to a method for treating a tissue opening, comprising: inserting a delivery device including first and second closing devices into a first portion of target tissue; deploying a first anchoring element of the first closing device out of the delivery device into the first portion of target tissue while retaining within the delivery device a first coupling portion of the first closing device, the first coupling portion being coupled to the first anchoring element via a first tether; withdrawing the delivery device from the first portion of target tissue; deploying the first coupling portion of the first closing device outside the first portion of target tissue; inserting the delivery device into a second portion of target tissue separated from the first portion of target tissue; deploying a second anchoring element of the second closing device out of the delivery device into the second portion of target tissue while retaining within the delivery device a second coupling portion of the second closing device, the second coupling portion being coupled to the second anchoring element via a second tether; withdrawing the delivery device from the second portion of target tissue; deploying the second coupling portion of the second closing device outside the second portion of target tissue; and coupling the first and second coupling portions to one another to draw the draw the first and second portions of target tissue toward one another and to maintain the first and second portions of target tissue in desired positions relative to one another.

In an embodiment, the first and second coupling portions may be magnetic.

In an embodiment, the system may further comprising a handle coupled to a proximal end of the delivery device and an actuation mechanism for incrementally deploying the first and second closing devices from the delivery device.

In an embodiment, the actuation mechanism may include an actuator on the handle coupled to an actuation member slidably received in the delivery device to expel the first and second closing devices out of the delivery device upon operation of the actuator.

In an embodiment, each of the first and second closing devices may include a projection extending transversely to an axis of the tether to resist removal of each of the first and second anchoring elements from tissue into which it has been inserted.

### BRIEF DESCRIPTION OF THE DRAWINGS

FIG. 1 shows a perspective view of a system according to a first exemplary embodiment of the present disclosure;

FIG. 2 shows another perspective view of the system of FIG. 1;

FIG. 3 shows another perspective view of the system of FIG. 1;

FIG. 4 shows a perspective view of a system according to a second exemplary embodiment of the present disclosure;

FIG. 5 shows another perspective view of the distal portion of the delivery device of FIG. 4;

FIG. 6 shows yet another perspective view of a distal portion of a delivery device of FIG. 4;

FIG. 7 shows a perspective view of a system according to an alternate embodiment of the present disclosure; and

FIG. 8 shows another perspective view of the system of FIG. 6.

### DETAILED DESCRIPTION

The present disclosure may be further understood with reference to the following description and appended drawings, wherein like elements are referred to with the same reference numerals. The present disclosure is directed to devices for the treatment of tissue and, in particular endoscopic tissue treatment devices. Exemplary embodiments of the present disclosure describe anchors positioned about a periphery of a tissue perforation and drawn toward one another via magnets to close the tissue opening. It should be noted that the terms “proximal” and “distal” are intended to refer to a direction toward (proximal) and away from (distal) a user of the device.

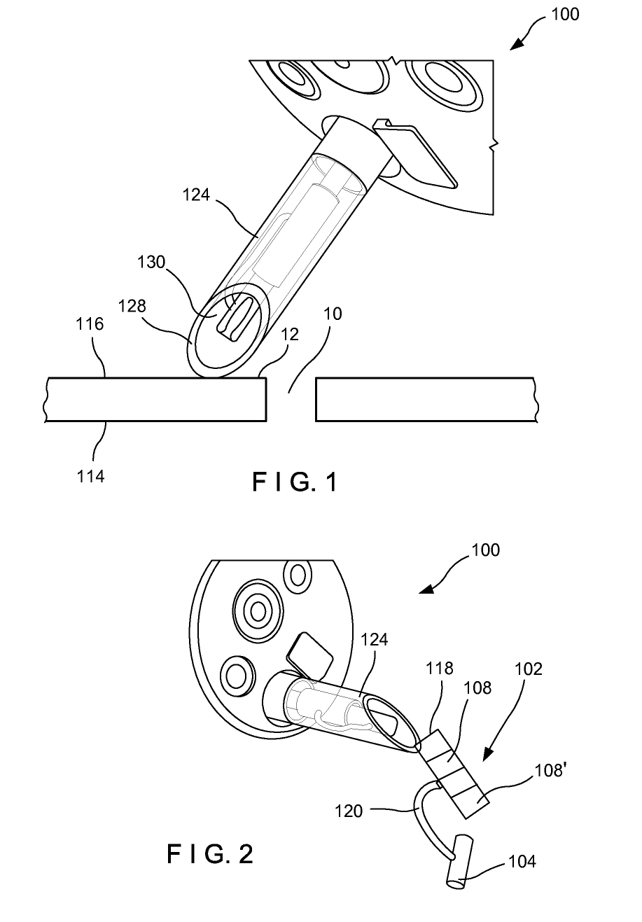

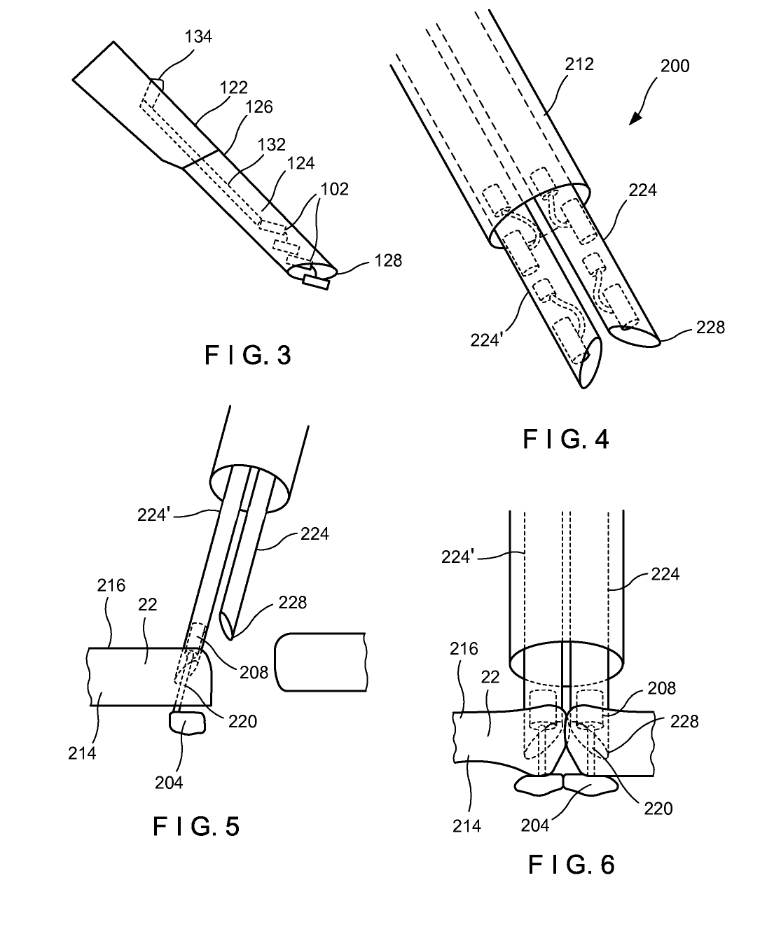

As shown in FIGS. 1-3, a system 100 for treating tissue according to an exemplary embodiment of the present disclosure comprises a handle 122 coupled to the proximal end 126 of a longitudinal sheath 124 including a lumen 130 extending there through, sized and shaped to receive a plurality of T-tags 102. The handle 122 includes an actuator 134 coupled to an actuation rod 132 slidably inserted in the lumen 130. A distal end of the rod 132 contacts the proximal end of a proximal-most one of the T-tags 102 which abuts a more distal T-tag 102 and so on to a distal-most one of the T-tags 102. The actuator 134 is coupled to the rod 132 by a known mechanism so that a first actuation of the actuator 134 moves the rod 132 by a known distance to sequentially deploy an anchor 104 of the distal-most T-tag 102 and, subsequently as will be described below, a second actuation separately deploys a magnet 108 of the distal-most T-tag 102. Each T-tag 102 comprises an anchor 104 at a distal end and a magnet 108 at a proximal end thereof connected to one another by a tether 120. The sheath 124 extends to a tissue piercing distal end 128 and is sized and shaped for insertion to a target location in the body via a delivery tool such as, for example, a flexible endoscope. Thus, the sheath 124 according to this embodiment is formed of a material having a flexibility sufficient to enable it to pass through the working channel of a flexible endoscope when that endoscope extends along a tortuous path into the body (e.g., within a natural body lumen). However, those skilled in the art will understand that this system 100 is completely compatible with insertion through rigid delivery tools and, in such cases, the sheath 124 may be rigid as well.

Each T-tag 102 is configured to be slidably received in the lumen 130 of the sheath 124 and to be deployed therefrom in target tissue along a periphery of a tissue opening 10. The anchors 104 may be biased laterally outward toward a T-shape configuration in which the anchor 104 extends transverse to the tether 120 so that, when embedded in or when passed through a portion of tissue, the anchor 104 resists removal from the tissue. When within the sheath 124, the anchors 104 are constrained by contact with an inner surface of the lumen 130 to remain in an insertion configuration in which they to extend substantially parallel to the tethers 120 to facilitate insertion thereof through the sheath 124. Once the anchor 104 has been moved out of the sheath 124, it is freed to revert to the anchoring T-shape configuration as described above.

Each of the anchors 108 is coupled to a magnet 108 via a tether 120 so that the magnet 108 extends from a proximal surface 116 of the tissue 12 in which the anchor 104 is embedded. In an exemplary embodiment, the magnet 108 may be cylindrical to facilitate its movement through the lumen 130. However, it will be apparent to those skilled in the art that various other configurations are possible. In an exemplary embodiment, the magnets 108 may be rare earth magnets as they exhibit a more powerful magnetic field in comparison with other magnets of the same size. The attractive force produced by the magnet 108 is preferably strong enough to draw portions of the target tissue 12 toward one another sufficiently to seal a perforation to hold the perforation closed during the body's natural healing processes. It will be apparent to those having skill in the art that the attractive force produced by the magnet 108 may be based on the size of the magnet 108 that will fit through the endoscope working channel and standard sizes offered. For example, a small diametrically magnetized magnet having a diameter of 0.062 inches (0.157 cm) and length of 0.125 inches (0.318 cm) may have a pull force of 0.15 pounds (0.07 kg). In another example, the large diametrically magnetized magnet having a diameter of 0.125 inches (0.318 cm) and length of 0.5 inches (1.27 cm) may have a pull force of 1.2 pounds (0.544 kg). Although the potency of rare earth magnets does decrease over time, this loss is negligible over the time needed for natural closure. In addition, although temperatures above a maximum operating temperature can also lower the power of rare earth magnets and temperatures above the Curie temperature can completely eliminate their magnetic properties, these temperatures are well above the temperatures to which the magnets 108 will be exposed in the body. For example, the maximum operating temperatures are generally greater than 80 degrees Celsius and the Curie Temperature is generally 1000 degrees Celsius or more. The magnets 108 may include a casing 118 surrounding all or part of the magnet 108 and coupling the magnet 108 to the tether 120. The casing 118 may simplify manufacturing as drilling through magnets may be difficult and may expose potentially toxic materials. That is, by trapping the tether 120 against the magnet 108, the casing 118 permits the binding of the tether 120 to the magnet 108 without requiring a hole in the magnet. Furthermore, the casing 118 may be used to accommodate a magnet 108 formed of multiple, mechanically separate magnet segments 108′. That is, the casing 118 may hold multiple magnet segments 108′ together without the need to couple each segment 108′ to the tether 120. Finally, the tether 120 may be made of any suitably strong biocompatible material and may, optionally be bioabsorbable as would be understood by those skilled in the art. A bioabsorbable tether 120 permits the magnets 108 to be left in the body so that, after a predetermined time has passed and the tether 120 has degraded, the magnets 108 are released to pass naturally out of the body. Those skilled in the art will understand that the tether 120 must be constructed to hold the magnets 108 to their respective anchors 104 for a time period sufficient to permit natural healing processes to progress to the point at which the tissue surrounding the opening 10 has bonded together sufficiently to hold itself together sealing the wound.

In use, the endoscope is inserted into the body (e.g., through a natural body lumen accessed via a natural body orifice) to a position adjacent a tissue opening 10 to be closed. The sheath 124 is then advanced distally out of the endoscope so that the distal end 128 penetrates a first portion of the tissue 12 adjacent to the opening 10. The actuator 134 is then operated to move the anchor 104 of the distal-most T-tag 102 out of the sheath 124. Upon exiting the sheath 124, the anchor 104 reverts to the T-shaped configuration to lock the anchor within the tissue 12 while the magnet of this T-tag 102 remains within the lumen 130. The sheath 124 is then retracted proximally out of the tissue 12 and the actuator 134 is operated again to deploy the magnet 108 on the opposite side of the tissue 12 with the tether 120 extending through the tissue 12. The endoscope may then be moved to a second portion of tissue at which it is desired to place a second T-tag 102 and the implantation procedure is repeated for this subsequent T-tag 102. As the T-tags 102 are implanted, the attractive force of the magnets 108 draws the T-tags 102 toward one another, pulling the edges of the tissue perforation 10 toward one another to close the tissue perforation. As would be understood by those skilled in the art, the number and placement of the T-tags 102 required to seal an opening 10 will depend on factors such as the type of tissue, the surrounding anatomy and the geometry of the opening 10.

In addition, depending on the tissue properties and the spacing between the magnets 108 it may be necessary to draw the tissue together manually to initially couple the magnets 108. For example, after a first T-tag 102 has been implanted, a user may wish to use the sheath 124 to draw the second magnet 108 toward the first magnet 108. That is, after the anchor 104 of the second T-tag 102 been implanted but before the magnet 108 of the second T-tag 102 has been ejected from the sheath 124, the distal end 128 of the sheath 124 may be moved toward the magnet 108 of the first T-tag 102 (e.g., by maneuvering the endoscope) to drag the tissue in which the anchor 104 of the second T-tag 102 is embedded toward the first T-tag 102. The magnet 108 of the second T-tag 102 may then be deployed from the sheath 124 next to the magnet 108 of the first T-tag 102 to ensure that the magnetic force draws these T-tags 102 (and the tissue to which they are coupled) toward one another.

In an alternative embodiment, one or more T-tags 102 may include a proximal member including a Velcro or hook and loop portion which, when brought into contact with a complementary Velcro or hook and loop portion of a proximal member of another T-tag 102 will couple the T-tags 102 to one another. As would be understood by those skilled in the art, this Velcro may be placed either on a magnet 108 as described above to enhance the coupling or may be on a non-magnetic proximal member to form the only coupling with other similar proximal members. Alternatively, one or more of the anchors 104 may include a Velcro portion for similar coupling to other similar anchor members. As the Velcro does not provide an attractive force until mechanically contacted by a corresponding Velcro structure, where the Velcro is included on non-magnetic members, the items will need to be manipulated to bring them into contact with one another. It will be understood by those of skill in the art that the T-tag 102 may be sealed via other methods.

As shown in FIGS. 4-6, a system 200 according to another exemplary embodiment is substantially similar to the system 100, comprising a plurality of T-tags 202 to be placed in target tissue 22 about a periphery of a tissue perforation 20 via a delivery tool 212 except that the system 200 includes two sheaths 224, 224′ adjacent to one another within a surrounding member 212. Each sheath 224, 224′ may be substantially similar to the longitudinal sheath 124 described above in regard to system 100 each including an independent actuation mechanism (actuation rod and actuator on handle) as described above in regard to the system 100 and a plurality of T-tags 102. The T-tags 202 may be made smaller to reduce the size of the sheaths 224, 224′ to keep the entire profile of the system 200 small enough to allow the surrounding member 212 to be slidably received in the working channel of an endoscope. The dual sheath arrangement of the system 200 permits the T-tags 202 to be more easily coupled to one another. For example, after an anchor 204 of a first T-tag 202 has been deployed from the first sheath 224 in tissue 12 by the same method described above in regard to the system 100, the second sheath 224′ may be positioned over a second portion of tissue into which the next T-tag is to be deployed while retaining the magnet 208 of the first T-tag 202 within the first sheath 224. The second sheath 224′ is then inserted into the second portion of target tissue 22 and the anchor of a second T-tag 202 in the second sheath 224′ is inserted into the target tissue 22. The second sheath 224′ is then withdrawn from the target tissue 22 and the magnets 208 of the first and second T-tags 102 may be deployed from the respective sheaths 224, 224′ adjacent to one another with the first and second portions of tissue drawn together. Subsequent T-tags 202 may then be deployed in a similar manner to close the tissue opening.

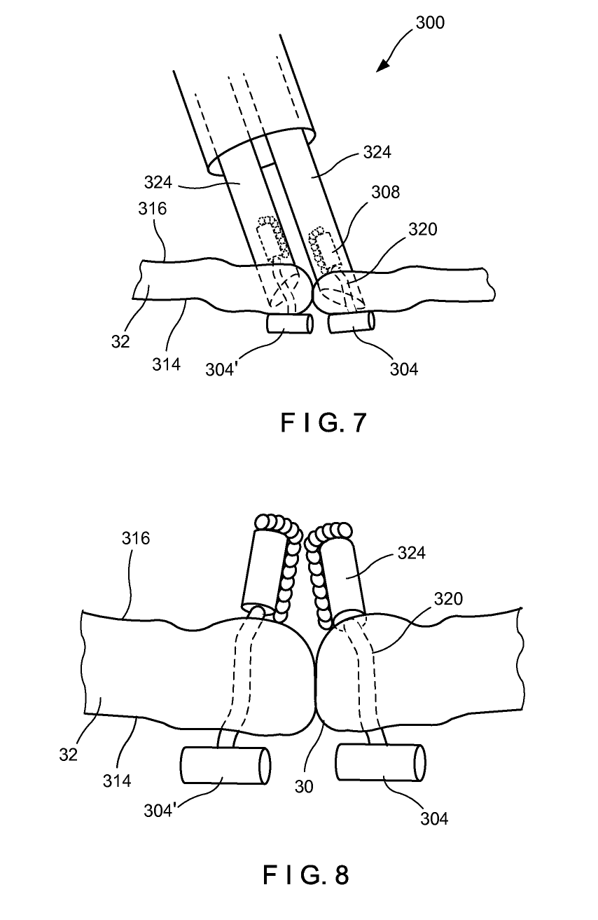

As shown in FIGS. 7 and 8, a system 300 according to another exemplary embodiment is substantially similar to the system 100 except that the proximal part (corresponding to the magnets 108) of each T-tag 302 is configured to mechanically couple to the proximal part of another of the T-tags 302. As those skilled in the art will understand, this mechanical coupling may be in addition to, or completely separate from any magnetic coupling and may comprise any number of mechanically interlocking shapes. In the exemplary embodiment shown in FIGS. 7 and 8, the T-tags 302 are to be placed in target tissue 32 about a periphery of a tissue perforation 30 via a delivery tool 312. The delivery tool 312 is substantially similar to the delivery tool (112) and the T-tags 302 are stacked in the sheath 324 in a similar manner. Those skilled in the art will understand that for certain mechanical connections it may only be possible for a first coupling element of a first configuration to couple to a second coupling element having a complementary configuration. In these cases it is important to ensure that the T-tags are deployed in the proper order. For example, in a single sheath system, each T-tag 302 is followed by the opposite connection, i.e. key, lock, key, lock, etc. as you move proximally into the sheath 324 from the distal end. In a system using two sheaths it might be desired to place all T-tags 302 including coupling elements of the first configuration in one of the sheaths and the T-tags 302 including the complementary elements in the other sheath. The sheath 324 would then be manipulated as described above to mechanically connect the coupling elements of the T-tags 302 to one another.

It will be apparent to those skilled in the art that various modifications may be made in the present disclosure, without departing from the spirit or scope of the disclosure. Thus, it is intended that the present disclosure cover modifications and variations of this disclosure provided that they come within the scope of the appended claims and their equivalents.

What is claimed is:

1. A closing device for treating tissue, comprising:
an anchoring element at a distal end thereof for anchoring the device in a target portion of tissue and a coupling element, the anchoring element and the coupling element being coupled to one another by a tether so that, when the anchoring element is anchored in a target portion of tissue, the tether passes through the portion of tissue and the coupling element remains outside the portion oft issue, wherein the closing device is configured to couple to a second closing device to hold the portion of tissue in a desired location relative to one another.

2. The closing device of claim 1, wherein the coupling element is magnetic.

3. A system for treating tissue, comprising;
a first closing device including a first anchoring element at a distal end thereof for anchoring the first closing device in a first target portion of tissue and a first coupling element, the first anchoring element and the first coupling element being coupled to one another by a first tether so that, when the first anchoring element is anchored in a first target portion of tissue, the first tether passes through the first portion of tissue and the first coupling element remains outside the first portion of tissue;
a second closing device including a second anchoring element at a distal end thereof for anchoring the second closing device in a second target portion of tissue and a second coupling element, the second anchoring element and the second coupling element being coupled to one another by a second tether so that, when the second anchoring element is anchored in a second target portion of tissue, the second tether passes through the second portion of tissue and the second coupling element remains outside the second portion of tissue;
the first and second coupling elements being configured to couple to one another to hold the first and second portions of tissue in a desired position relative to one another.

4. The system of claim 3, wherein each of the first anchoring elements includes a projection extending transversely to an axis of the tether to resist removal of the first anchoring element from tissue into which it has been inserted.

5. The system of claim 3, wherein the first and second coupling elements are magnetic.

6. The system of claim 4, further including a first sheath with first tissue piercing distal tip and defining a first lumen therein sized and shaped to receive at least one of the first and second closing devices.

7. The system of claim 6, wherein the first closing device includes a casing surrounding at least a portion of the first coupling element and a portion of the first tether to couple the first tether to the first coupling element.

8. The system of claim 6, further comprising a handle coupled to a proximal end of the first sheath and a first actuation mechanism for incrementally deploying the first closing element from the first sheath.

9. The system of claim 8, wherein first actuation mechanism includes a first actuator on the handle coupled to a first actuation member slidably received in the first sheath to expel the first closing device out of the first sheath upon operation of the first actuator.

10. The system of claim 6, wherein the delivery tool includes a second sheath with a second tissue piercing distal tip and defining a second lumen therein sized and shaped to receive the second closing device while the first closing device is received within the first sheath.

11. The system of claim 9, wherein at least the first and second lumens includes a plurality of further closing devices, each of the further closing devices including a further anchoring element at a distal end thereof for anchoring the further closing device in a further target position of tissue and a further coupling element, each further anchoring element being coupled to a corresponding further coupling element by an associated further tether so that, when each further anchoring element is anchored in tissue, the associated further tether passes through the tissue and the corresponding further coupling element remains outside the tissue.

12. The system of claim 8, further comprising a second actuation mechanism including a second actuator on the handle coupled to a second actuation member slidably received in the second sheath to push the second closing device out of the second sheath upon operation of the second actuator.

13. The system of claim 8, wherein the first coupling element includes a first hook and loop portion and the second coupling element includes a second hook and loop portion configured to couple to the first hook and loop portion.

14. The system of claim 3, wherein the first and second coupling elements are shaped to mechanically lock to one another.

15. A method for treating a tissue opening, comprising:
inserting a delivery device including first and second closing devices into a first portion of target tissue;
deploying a first anchoring element of the first closing device out of the delivery device into the first portion of target tissue while retaining within the delivery device a first coupling portion of the first closing device, the first coupling portion being coupled to the first anchoring element via a first tether;
withdrawing the delivery device from the first portion of target tissue;
deploying the first coupling portion of the first closing device outside the first portion of target tissue;
inserting the delivery device into a second portion of target tissue separated from the first portion of target tissue; deploying a second anchoring element of the second closing device out of the delivery device into the second portion of target tissue while retaining within the delivery device a second coupling portion of the second closing device, the second coupling portion being coupled to the second anchoring element via a second tether;
withdrawing the delivery device from the second portion of target tissue;
deploying the second coupling portion of the second closing device outside the second portion of target tissue; and
coupling the first and second coupling portions to one another to draw the draw the first and second portions of target tissue toward one another and to maintain the first and second portions of target tissue in desired positions relative to one another.

16. The method of claim 15, wherein the first and second coupling portions are magnetic.

17. The method of claim 15, wherein the first and second closing devices are incrementally deployed from the delivery device via a handle coupled to a proximal end of the delivery device, wherein the handle includes an actuation mechanism configured to incrementally deploy the first and second closing devices.

18. The method of claim 15, wherein the actuation mechanism includes an actuator on the handle coupled to an actuation member slidably received in the delivery device to expel the first and second closing devices out of the delivery device upon operation of the actuator.

19. The method of claim 15, wherein each of the first and second closing devices includes a projection extending transversely to an axis of the tether to resist removal of each of the first and second anchoring elements from tissue into which it has been inserted.
